(19) United States
(12) Patent Application Publication
(10) Pub. No.: US 2006/0003407 A1
(43) Pub. Date: Jan. 5, 2006

(54) Methods for preparing highly active april ligand polypeptides

(75) Inventors: Paul D. Rennert, Holliston, MA (US); Graham K. Farrington, Acton, MA (US); John K. Eldredge, So. Chatham, MA (US); Marc Pelletier, Jamaica Plain, MA (US); Yen-Ming Hsu, Lexington, MA (US)

Correspondence Address: FISH & NEAVE IP GROUP;ROPES & GRAY LLP, 1251 AVENUE OF THE AMERICAS FL C3, NEW YORK, NY, 10020-1105, US

(73) Assignee: Biogen Idec MA Inc.

(21) Appl. No.: 11/084,647

(22) Filed: Mar. 17, 2005

Related U.S. Application Data
(63) Continuation of application No. PCT/US03/29178, filed on Sep. 19, 2003.
(60) Provisional application No. 60/412,467, filed on Sep. 19, 2002.

(51) Int. Cl. C07H 21/04; C12P 21/06; C07K 14/715

(52) U.S. Cl. 435/69.1; 435/320.1; 435/325; 530/350; 536/23.5

(57) ABSTRACT

The present invention relates to improved methods for producing biologically active truncated APRIL ligand polypeptides and analogs thereof. The invention further relates to truncated APRIL ligand polypeptides and analogs thereof that retain a high biological activity and may be isolated in high yields, as well as the nucleotide sequences that encode the truncated APRIL ligand polypeptides and analogs thereof. The invention also relates to compositions of the biologically active truncated APRIL ligand polypeptides and analogs thereof. The invention further relates to the use of the biologically active truncated APRIL ligand polypeptides and analogs thereof in promoting cell proliferation.

### TECHNICAL FIELD OF THE INVENTION

The present invention relates to improved methods for producing biologically active truncated APRIL ligand polypeptides and analogs thereof. The invention further relates to truncated APRIL ligand polypeptides and analogs thereof that retain a high biological activity and may be isolated in high yields, as well as the nucleotide sequences that encode the truncated APRIL ligand polypeptides and analogs thereof. The invention also relates to compositions of the biologically active truncated APRIL ligand polypeptides and analogs thereof. The invention further relates to the use of the biologically active truncated APRIL ligand polypeptides and analogs thereof in promoting cell proliferation.

### BACKGROUND OF THE INVENTION

Members of the Tumor Necrosis Factor (TNF) family of ligands, so named for their structural similarity to TNF-α, are key components in diverse processes, such as cell growth, fetal development, inflammatory responses, cellular immunity and apoptosis. TNF ligands may act locally as type II membrane-bound proteins through direct cell-to-cell contact or as secreted proteins having autocrine, paracrine or endocrine functions. TNF family members bind TNF receptor (TNF-R) family members via their C-terminal extracellular domain. Various TNF ligands and receptors include TNF, lymphotoxins (LT), Fas, CD27, CD30, CD40, 4-1BB, OX-40, TRAMP, CAR-1, TRAIL, GITR, HVEM, osteoprotegrin, NGF, TRAIN, BAFF, APRIL and TWEAK. The structure of TNF family members has been well-defined by crystallographic analysis. The quaternary structures of the TNF family members have been shown to exist as trimers by analysis of their crystal structures. This propensity to assemble into oligomeric complexes may be important in the formation of the receptor binding site.

A defining feature of this family of cytokine receptors is found in the cysteine rich extracellular domain, initially revealed by the molecular cloning of two distinct TNF receptors. This family of genes encodes glycoproteins characteristic of Type I transmembrane proteins having an extracellular ligand binding domain, a single membrane spanning region and a cytoplasmic region involved in activating cellular functions. The cysteine-rich ligand binding region exhibits a tightly knit disulfide linked core domain, which, depending upon the particular family member, is repeated multiple times. Most receptors have four domains, although there may be as few as one, or as many as six.

TNF family members play a role in the regulation of the immune system, controlling cell survival and differentiation, as well as acute host defense systems, such as inflammation. Continued efforts in the art to manipulate members of the TNF family for therapeutic benefit may provide unique means to control disease. For instance, some of the ligands of this family can directly induce the apoptotic death of many transformed cells, e.g., LT, TNF, Fas ligand and TRAIL. Fas and possibly TNF and CD30 receptor activation can induce cell death in nontransformed lymphocytes which may display an immunoregulatory function.

The ability to induce programmed cell death is an important and well-studied feature of several members of the TNF family. Fas mediated apoptosis appears to play a role in the regulation of autoreactive lymphocytes in the periphery and possibly the thymus. Death in these cell lines in response to, for example, TNF or Fas signaling, displays features of apoptosis.

The TNF family of ligands may be categorized into three groups based on their ability to induce cell death. First, TNF, Fas ligand and TRAIL can efficiently induce cell death in many cell lines and their receptors most likely have good canonical death domains. Presumably the ligand to DR-3 (TRAMP/WSL-1) would also fall into this category. Next there are those ligands, such as TWEAK, CD30 ligand, and LTα1β2, which trigger a weaker death signal limited to a few cells. Studies in these systems have suggested that a separate weaker death signaling mechanism exists. Lastly, there are those members that cannot efficiently deliver a death signal. All groups may exert antiproliferative effects on some cell types as a consequence of inducing cell differentiation, e.g., CD40.

In general, death is triggered following the aggregation of death domains which reside on the cytoplasmic side of the TNF receptors. The death domain orchestrates the assembly of various signal transduction components which lead to activation of the caspase cascade. Some receptors lack canonical death domains, e.g. LT-β receptor and CD30, yet can induce cell death, albeit more weakly. Conversely, signaling through other pathways such as CD40 is required to maintain cell survival. There remains a need to further identify and characterize the functions of the TNF family members, thereby facilitating the development of new therapies for TNF family-related diseases.

APRIL (a proliferation-inducing ligand, also known as TALL-2 and TRDL-1α) is a ligand of the TNF family, and a positive regulator of cell proliferation and tumor growth. See Ware J. Exp. Med. 192:F35-38 (2000). APRIL ligand is overexpressed in various types of human malignancies, and its ectopic expression directly correlates with elevated tumorigenecity in a fibrosarcoma model. See Ware J. Exp. Med. 192:F35-38 (2000). Moreover, APRIL ligand binding has been detected in cell lines from both lymphoid and nonlymphoid origin, which may express different APRIL receptors. See Ware J. Exp. Med. 192:F35-38 (2000). Antagonizing APRIL ligand dramatically slows tumor growth in a murine xenograft model. See Rennert et al. J. Exp. Med. 11:1677-1683 (2000). Taken together, these studies suggest that APRIL ligand supports normal cell proliferation or survival functions, and that this function is co-opted by cancer ells during tumorigenesis.

Like other TNF family members, APRIL ligand is synthesized as a type II transmembrane precursor, but is cleaved between the transmembrane and receptor binding domains by a furin convertase. APRIL ligand processing takes place in the Golgi apparatus prior to its secretion, which suggests that APRIL ligand must interact with its receptors as a soluble cytokine, rather than through cell to cell interactions. See Lopez-Fraga et al. EMBO Rep. 2:945-951 (2001). The receptor-binding domain on the APRIL ligand protein is 33% identical to that of another furin-cleaved TNF-family ligand called BAFF (B lymphocyte activation factor of the TNF family, also known as BlyS/zTNF4/TALL-1/THANK). See Ware J. Exp. Med. 192:F35-38 (2000). BAFF is a potent growth factor for B cells that is critical for B cell growth and survival. See Rolink et al. Curr Opin Immunol. 14:266-275 (2002). APRIL ligand also shares significant homology with two other ligands with furin-cleavage sites, TWEAK and EDA. See Bodmer et al. Trends Biochem Sci. 27:19-26 (2002).

The two APRIL receptors known to date are TACI (Transmembrane activator and calcium modulator and cyclophilin interactor) and BCMA (B cell maturation antigen). See Cao et al. Cell 107:763-775 (2001). These receptors are expressed only by lymphoid cells. Therefore, APRIL ligand is distinguished from BAFF by its ability to bind to nonlymphoid cells. Although BAFF also binds TACI and BCMA, BAFF appears to mediate its B cell activities primarily through the BAFF receptor (BAFF—R), which does not bind the APRIL ligand. See Rolink et al. Curr Opin Immunol. 14:266-275 (2002). In addition, evidence suggests that a third specific receptor for the APRIL ligand exists. TACI and BCMA mRNAs are not detectable in the murine NIH-3T3 fibroblastic cell line or in human HT-29 adenocarcinoma cells, even though these cells bind to truncated APRIL ligand, but not BAFF. See Rennert et al. J. Exp. Med. 192:1677-1684 (2000).

Constitutive APRIL ligand expression in tumor cells shows that it may be an important “tumor growth factor”. See Hahne et al. J. Exp. Med. 188:1185-1190 (1998). APRIL expression in solid tumor tissue, along with observations that such tumor cells bind APRIL ligand, suggests that this cytokine can create an autocrine growth signal that results in the increased tumorigenecity or survival of these cells. Overexpression studies demonstrated that APRIL ligand is a positive regulator of cell growth. See Ware J. Exp. Med. 192:F35-38 (2000).

Obtaining high levels of a purified, active APRIL ligand has been difficult due to its severe aggregation and insolubility. The increasing importance of APRIL ligand in the regulation of cellular growth, cancer, and immunological processes has prompted a need for improved methods of producing biologically active APRIL ligand polypeptides for both therapeutic and research purposes.

### SUMMARY OF THE INVENTION

The invention provides improved methods for producing biologically active truncated APRIL ligand polypeptides or analogs thereof comprising the steps of: (a) providing a vector comprising a nucleotide sequence encoding a truncated APRIL ligand polypeptide operably linked to an expression control sequence, (b) introducing the vector into a host cell, (c) growing the host cell in a culture medium under conditions which allow the truncated APRIL ligand polypeptide or analog thereof to be expressed and secreted into the culture medium, (d) separating the culture medium from the host cell, (e) subjecting the separated culture medium to adsorption chromatography; and (f) recovering truncated APRIL ligand polypeptide or analog thereof fractions. The invention also provides truncated APRIL ligand polypeptides and analogs thereof produced by the methods disclosed herein, that retain a high biological activity and may be isolated in high yields, as well as the nucleotide sequences that encode the truncated APRIL ligand polypeptides and analogs thereof. The invention further provides compositions comprising the biologically active truncated APRIL ligand polypeptides and analogs thereof. The invention further provides methods of promoting cell proliferation by administering the biologically active truncated APRIL ligand polypeptides and analogs thereof.

### BRIEF DESCRIPTION OF THE DRAWINGS

FIG. 1 depicts the characterization of the purified truncated murine APRIL ligand polypeptide. Purified, myc-tagged, truncated murine APRIL ligand polypeptide (amino acids 106-241 of SEQ ID NO: 5) was expressed in Pichia pastoris, purified as described herein, and 6 μg was analyzed on reducing (lane 1) and non-reducing (lane 2) polyacrylamide gels. Molecular weight markers in kilodaltons (Kd) are shown on the left.

FIG. 2 depicts the characterization of the purified truncated human APRIL ligand polypeptide (amino acids 115-250 of SEQ ID NO: 6). Analysis by non-reducing SDS-PAGE of the purified, FLAG-tagged, truncated human APRIL ligand polypeptide (amino acids 115-250 of SEQ ID NO: 6) reveals two bands of 21 and 22 kilodaltons in size, most likely representing glycosylated and non-glycosylated forms of the protein.

FIG. 3 depicts the purification of truncated human APRIL ligand polypeptide (amino acids 115-250 of SEQ ID NO: 6) on Superdex 200. (A) depicts the elution profile of truncated human APRIL ligand polypeptide (amino acids 115-250 of SEQ ID NO: 6) on Superdex 200. (B) depicts SDS-PAGE analysis of the fractions from the Superdex 200 column. FLAG-tagged truncated human APRIL ligand polypeptide (amino acids 115-250 of SEQ ID NO: 6) eluted as a 60 kilodalton trimeric protein. Molecular weight standards in kilodaltons (Kd) were used as a comparison (shown on the right).

FIG. 4 is a graphical representation of the results of FACs analysis of the binding of truncated murine APRIL ligand polypeptide to the APRIL receptor, BCMA. FACs analysis demonstrated that the truncated murine APRIL ligand polypeptide specifically binds APRIL receptors. The inability of an inactive human FLAG-tagged BAFF ligand to bind to the APRIL receptor was used as a negative control.

FIG. 5 depicts the results of an immunoprecipitation of FLAG-tagged truncated human APRIL ligand polypeptide (amino acids 115-250 of SEQ ID NO: 6) using Protein A followed by Western analysis using anti-FLAG M2 antibody. Purified truncated human APRIL ligand polypeptide (amino acids 115-250 of SEQ ID NO: 6) specifically binds to BCMA-Fc but not to BAFF-Fc. FLAG-tagged truncated human APRIL ligand polypeptide (amino acids 115-250 of SEQ ID NO: 6), either purified (FLAGhuAPRIL purified) or unpurified (FLAGhuAPRIL SN), bound specifically to BCMA (BCMA-Fc) but not to the BAFF receptor (BAFFR-Fc). BAFF, which bound to both BCMA and BAFFR was used as an internal control.

FIG. 6 depicts the results of a binding analysis using various truncates of human APRIL ligand polypeptides. FACs analysis was used to demonstrate that only the shorter truncates of human APRIL ligand polypeptide (amino acid residues 115-250 of SEQ ID NO: 6), as opposed to a longer truncate (residues amino acids 105-250 of SEQ ID NO: 6), was able to completely and specifically bind to TACI+ in IM9 cells. This was demonstrated by the ability of a soluble competitor receptor (BCMA-Ig) to selectively block the binding of the shorter truncates of human APRIL ligand polypeptide (amino acid residues 115-250 of SEQ ID NO: 6) but not the longer truncate (amino acids residues 105-250 of SEQ ID NO: 6) to TACI+IM9 cells.

FIG. 7 depicts non-reducing and reducing PAGE analysis of the various truncates of the human APRIL ligand polypeptide. Analysis of three different truncates of the human APRIL ligand polypeptide comprising amino acid residues 105-250 of SEQ ID NO: 6, 110-250 of SEQ ID NO: 6 or 115-250 of SEQ ID NO: 6 on a non-reducing SDS-PAGE gel revealed that the longer truncates (i.e., those encoded by amino acid residues 105-250 of SEQ ID NO: 6 or amino acid residues 110-250 of SEQ ID NO. 6) formed high molecular weight aggregates, whereas the shorter truncates (i.e., those encoded by amino acid residues 115-250 of SEQ ID NO: 6) did not. Under reducing conditions, only the monomer and non-reducible forms of the molecules are present. Only the shortest form of the human APRIL ligand polypeptide (encoded by amino acid residues 115-250 of SEQ ID NO: 6) lacked high molecular weight aggregates under both non-reducing or reducing conditions.

FIG. 8 demonstrates that the purified truncated murine APRIL ligand polypeptide (amino acids 106-241 of SEQ ID NO: 5) induces cell proliferation. Cells were plated at 5×10³ cells/well in 96-well plates and synchronized by serum starvation for 16 hours before adding increasing concentrations of truncated murine APRIL ligand polypeptide (amino acids 106-241 of SEQ ID NO: 5) in low serum. The curve shows a dose dependent stimulation of NIH-3T3 cells using a purified truncated murine APRIL ligand polypeptide made using the methods disclosed herein. DNA synthesis was determined by [³H]-thymidine incorporation 24 hours after stimulation. The data points represent the mean values obtained from triplicate cultures and the error bars represent ±1 standard error of measurement (SEM).

FIG. 9 demonstrates that the purified truncated murine APRIL ligand polypeptide (amino acids 106-241 of SEQ ID NO: 5) potentiates an FGF-2 response. FIG. 9A is a graphical representation of the effect of increasing amounts of FGF-2 on NIH3T3 cell proliferation (as measured by [³H]-thymidine incorporation). FIG. 9B is a graphical representation of the effect of purified truncated murine APRIL ligand polypeptide in the presence or absence of 0.4 ng/mL FGF-2 in NIH-3T3 cell proliferation as measured by [³H]-thymidine incorporation.

FIG. 10 demonstrates the effect of trimeric APRIL ligand polypeptide induce-signaling. (A) NIH3T3 and HT29 cells were stimulated with increasing concentrations of truncated murine (amino acids 106-241 of SEQ ID NO: 5) or human (amino acids 115-250 of SEQ ID NO: 6) APRIL ligand polypeptides, respectively. The effect of increasing concentrations of the truncated polypeptides on the protein levels of IκB were measured by resolving cell lysates on SDS-PAGE followed by transferring to nitrocellulose membrane and probing with an antibody specific for IκB protein. To confirm equal protein loading, immunoblots were stripped and reprobed with an antibody against actin. (B) A graphical representation of the IκB observed in (A) normalized based on actin levels.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with the laboratory procedures and techniques of cell and tissue culture, molecular biology, immunology, microbiology, genetics, virology, protein chemistry, nucleic acid chemistry and hybridization described herein are to have the meanings as understood by those of ordinary skill in the art. The nomenclatures used in connection with the laboratory procedures and techniques of analytical chemistry, synthetic organic chemistry, and medicinal and pharmaceutical chemistry described herein are to have the meanings as understood by those of ordinary skill in the art.

The following terms, unless otherwise indicated, shall be understood to have the following meanings:

“Biologically active” refers to having an in vivo or in vitro activity which may be performed directly or indirectly.

“Comprises” or “comprising” refers to the inclusion of a stated integer or groups of integer but not the exclusion of any other integers or groups of integers.

“Expression control sequences” refer to sequences that allow for the constitutive or inducible expression of nucleotide sequences to which they are ligated under specific conditions or in specific cells. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. Examples of cellular processes that expression control sequences regulate include, but are not limited to, transcription, protein translation, messenger RNA splicing, immunoglobulin isotype switching, protein glycosylation, protein cleavage, protein secretion, intracellular protein localization and extracellular protein homing. The term “control sequences” is intended to include, at a minimum, all components whose presence is essential for expression and processing, and can also include additional components whose presence is advantageous, for example, leader sequences and fusion partner sequences. Expression control sequences may be of viral, bacterial, yeast, insect, or animal (including mammal, e.g., human) origin.

“Fusion protein” refers to a chimeric protein comprising amino acid sequences of two or more different proteins. Typically, a fusion protein results from in vitro recombinatory techniques well known in the art. However, a fusion protein may result from in vivo crossover or other recombinatory events. Typical examples of fusion partner moieties include, but are not limited to, toxic peptide moieties, complement proteins, radiolabeled proteins, cytokines, antibiotic proteins, and immunoglobulin fragments. These include, but are not limited to, immunoglobulin Fc fragment, myc tag, FLAG tag, His tag, albumin and ricin. Fusion partner moieties may be of animal (including mammal, e.g., human), bacterial, yeast, insect, or viral origin.

“Host cell” refers to a cell into which a recombinant expression vector has been introduced. It should be understood that such terms are intended to refer not only to the particular subject cell but to the progeny of such a cell. Host cells may be a bacterial, yeast, animal (including mammal, e.g., human) or insect cell. Because certain modifications may occur in succeeding generations due to either mutation or environmental influences, such progeny may not, in fact, be identical to the parent cell, but are still included within the scope of the term “host cell” as used herein.

“Isolated nucleic acid molecule” refers to a nucleic acid molecule (DNA or RNA) that has been removed from its native environment. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

“Isolated protein” or “isolated polypeptide” refer generally to a protein or polypeptide that by virtue of its origin or source of derivation: (1) is substantially free of naturally associated components that accompany it in its native state, (2) is substantially free of other proteins from the same species (3) is expressed by a cell from a different species, or (4) does not occur in nature. Thus, a polypeptide that is chemically synthesized, synthesized in a cell-free biological system (e.g., a rabbit reticulocyte lysate), or synthesized in a cellular system different from the cell from which it naturally originates will be “isolated” from its naturally associated components. A protein may also be rendered substantially free of naturally associated components by isolation, using protein purification techniques as disclosed herein or as is otherwise known in the art.

“Nucleic acid molecule” refers to either ribonucleotides (RNA) or deoxynucleotides (DNA) or a modified form of either type of nucleotide. The term includes single and double stranded forms of DNA and may be produced or maintained in a bacterial, yeast, plant, animal (including mammal, e.g., human), or insect cell, or may be viral or formed by an in vitro synthesis technique.

“Operably linked” sequences include both expression control sequences that are contiguous with the gene of interest and expression control sequences that act in trans or at a distance to control the gene of interest.

“Polypeptide analogs” refer to polypeptides that are derived from truncated APRIL ligand polypeptides, but differs therefrom in their amino acid sequences. Polypeptides with changes in their amino acid sequences may be muteins or fusion proteins. Typically, polypeptide analogs comprise a conservative amino acid substitution (or insertion or deletion) with respect to the truncated APRIL ligand polypeptide sequences. Polypeptide analogs also refer to polypeptides that have non-amino acid sequence differences as compared with the truncated APRIL ligand polypeptides. These differences may be chemical or biochemical, and include, but are not limited to, the types of modifications specifically disclosed herein.

“Subjects” are humans and non-human subjects. An example of a subject is a human patient.

“Truncated APRIL ligand polypeptides” refer to APRIL ligand polypeptides that have an amino-terminal deletion. Truncated APRIL ligand polypeptides typically have an amino-terminal deletion resulting in a polypeptide encoded by amino acid residues 106-241 of SEQ ID NO: 5, amino acid residues ranging from amino acid 115-250 of SEQ ID NO: 6 to amino acid 133-250 of SEQ ID NO: 6. Accordingly, truncated APRIL ligand polypeptides may be polypeptides consisting essentially of amino acid residues X-250 of SEQ ID NO: 6, wherein X is any amino acid residue selected from amino acids 115 to 133. Truncated APRIL ligand polypeptides also include polypeptide analogs thereof and fusion proteins.

“Vectors” refer to DNA molecules that allow DNA sequences of interest to be cloned, propagated, recombined, mutated, or expressed outside of their native cells. Often vectors have expression control sequences that allow for the inducible or constitutive expression of gene sequences under specific conditions or in specific cells. Examples of vectors include, but are not limited to, plasmids, yeast artificial chromosomes (YACs), viruses, Epstein Bar Virus (EBV)-derived episomes, bacteriophages, cosmids and phagemids.

Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference.

Those who seek to express highly active proteins face many difficulties that typically are unique to each peptide sequence. One such difficulty is achieving high protein expression either in cell-based or cell-free expression systems. Furthermore, even when high levels of expression are attained, achieving high yields may be further complicated by peptide degradation, contamination, and protein inactivity caused by, e.g., lack of proper protein folding or post-translation modifications.

A major difficulty with many proteins is that, when expressed at high levels, they form insoluble aggregates. Insolubility leads to low specific activity and difficulties in purification. Protein insolubility often requires a denaturation and renaturation protocol, which typically subject the proteins to harsh pH conditions, further jeopardizing protein integrity and activity.

TNF ligand family members are characterized by a short N-terminal stretch of charged amino acids, often containing several lysine or arginine residues. Full-length APRIL ligand polypeptides contain an N-terminal lysine-rich region that leads to protein aggregation when expressed at high levels.

Methods of Producing Biologically Active Truncated APRIL Ligand polypeptide.

To overcome these obstacles, the present invention provides a method of producing a biologically active truncated APRIL ligand polypeptide or analog thereof which lacks the lysine rich region but retains the functional domains that are conservative across the various members of the TNF family of ligands. The method comprises the steps of: (a) providing a vector comprising a nucleotide sequence encoding a truncated APRIL ligand polypeptide operably linked to an expression control sequence; (b) introducing the vector into a host cell; (c) growing the host cell in a culture medium under conditions which allow the APRIL ligand polypeptide or analog thereof to be expressed and secreted into the culture medium; (d) separating the culture medium from the host cell; (e) subjecting the separated culture medium to adsorption chromatography; and (f) recovering APRIL ligand polypeptide or analog thereof fractions.

APRIL ligand polypeptides or analogs thereof may be expressed using techniques well known in the art. (See, e.g., Molecular Cloning A Laboratory Manual, 2^{nd} Ed., ed. by Sambrook et al. (Cold Spring Harbor Laboratory Press 1989) and Current Protocols in Molecular Biology, ed. by Ausubel et al. (Greene Publishing and Wiley Interscience, New York 1998), the contents of which are herein incorporated by reference). Expression vectors are well known in the art. Examples of vectors include, but are not limited to, plasmids, yeast artificial chromosomes (YACs), viruses, Epstein Bar Virus (EBV)-derived episomes, bacteriophages, cosmids and phagemids. In one embodiment, the vector is derived from the plasmid pIC9. In another embodiment, the vector is derived from the plasmid pCR3.

Expression control sequences are also well known in the art. Examples of cellular processes that expression control sequences regulate include, but are not limited to, gene transcription, protein translation, messenger RNA splicing, immunoglobulin isotype switching, protein glycosylation, protein cleavage, protein secretion, intracellular protein localization and extracellular protein homing. Often, expression control sequences cause inducible or constitutive expression of gene sequences under specific conditions or in specific cells.

In a preferred embodiment, expression control sequences that control transcription of the DNA encoding the truncated APRIL ligand polypeptide or analog thereof include, e.g., promoters, enhancers, transcription termination sites, locus control regions, RNA polymerase processivity signals, and chromatin remodeling elements. In another preferred embodiment, the expression control sequences regulate post-transcriptional events and include splice donor and acceptor sites and sequences that modify the half-life of the transcribed RNA, e.g., sequences that direct poly(A) addition or binding sites for RNA-binding proteins. In another preferred embodiment, the expression control sequences control translation and include ribosome binding sites, sequences which direct targeted expression of the polypeptide to or within particular cellular compartments, and sequences in the 5′ and 3′ untranslated regions that modify the rate or efficiency of translation. In another preferred embodiment, the expression control sequence is viral sequence, a bacterial sequence, a yeast sequence, an insect sequence, and a mammalian sequence. Thus, in one embodiment, the expression control sequence is a yeast sequence. In another embodiment, the expression control sequence is the methanol oxidase promoter.

The invention provides a host cell that has incorporated a vector comprising the nucleic acid molecules that encode the truncated APRIL ligand polypeptides or analogs thereof disclosed herein. In preferred embodiments, the host is a bacterial, yeast, animal (including mammal, e.g., human), or insect cell. In one embodiment, the host cell is a yeast cell. In another embodiment, the host cell is Pichia pastoris. In another embodiment, the host cell is a mammalian cell. In yet another embodiment, the mammalian cell is kidney 293T cells.

Once expressed at high levels, purifying proteins presents a challenging task. During the purification process, proteins are at risk of becoming inactive due to the many manipulations associated with biochemical purification schemes. Also, the proteins may be degraded by proteases that were carried over from the cell lysates. Furthermore, it may become difficult to remove contaminants such as other cellular proteins or other non-protein contaminants such as endotoxins, lipids, nucleic acids, and carbohydrates.

A typical starting point for protein purification is filtering debris from the expression system and removing lipids (typically using fibrofilters). Proteins are typically precipitated (or “salted out”) close to the beginning of the purification protocol. Various precipitation reagents may be used, including, but not limited to ammonia sulfate, acetone, methanol, and ethanol.

The type of chromatographic steps, and order of use, is critical to successful purification. For example, the proper pI ranges for the protein, hydrophobicity, the types of contaminants carried over from the expression system, the affinity of the proteins for the chromatographic matrix, complications that may result from eluting the proteins, protein size, any denaturing conditions required, and the effects of post-translational modifications must be considered.

Many chromatography platforms are available to those of skill in the art, and include adsorption chromatography, immunoaffinity, ion exchange (DEAE, Sepharose or carboxymethyl sepharose columns), HIC (hydrophobic interaction chromatography), phenyl sepharose, butyl sepharose, reverse phase high pressure liquid chromatography (HPLC), and gel filtration. (See, e.g., B. G. Belenkii and L. Z. Vilenchik, Modern Liquid Chromatography of Macromolecules, Elsevier Press, Amsterdam-Oxford-New York-Tokyo (1983); J. C. Giddings, Dynamics of Chromatography, Marcel Dekker, New York (1965); L. R. Snyder and J. J. Kirkland, Introduction to Modern Liquid Chromatography, Willey-Interscience, New York (1979); J. Porath and P. Flodin, Nature, 183, p. 1651 (1959); J. C. Moore, J. Polym. Sci., A, p. 835 (1964); J. A. Jonsson, Ed., Chromatographic Theory and Basic Principles, Marcel Dekker, New York (1987); J. Hermansson et al., in M. Ziefand and L. Crane, Eds., Chromatographic Chiral Separations, 40, pp. 245-81, Marcel Dekker, New York (1987), the teachings of these documents are incorporated herein by reference).

Adsorption chromatography includes, but is not limited to, such methods as affinity chromatography, ion exchange chromatography, dye ligand chromatography, immunoadsorbent chromatography and nonspecific adsorbent chromatography, including hydroxyapatite chromatography. (See, e.g., Scopes, R. K., Protein Purification: Principles and Practice, 2^{nd} Ed., Springer-Verlag, New York (1987), incorporated herein by reference).

In one embodiment, the adsorption chromatography is selected from the group consisting of hydroxyapatite chromatography and affinity chromatography. In a preferred embodiment, the adsorption chromatography is hydroxyapatite chromatography. In another preferred embodiment, the adsorption chromatography is affinity chromatography. In a another embodiment, the affinity chromatography is carried out using M1 Sepharose.

In one embodiment, the method further comprises subjecting the truncated APRIL ligand polypeptide and analog thereof fractions obtained following adsorption chromatography to size exclusion chromatography. Size exclusion matrices include, but are not limited to, dextrans, polyacrylamides, sepharose, agarose, cross-linked dextrans, cross-linked agarose, cross-linked polyacrylamide-agarose and ethylene glycol-methacrylate copolymers.

In a preferred embodiment, size exclusion chromatography is carried out using a size exclusion matrix capable of resolving proteins under 200 kilodaltons. In another preferred embodiment, the size exclusion chromatography is carried out using a size exclusion matrix capable of resolving proteins under 100 kilodaltons. In another preferred embodiment, the size exclusion chromatography is carried out using a size exclusion matrix capable of resolving proteins under 75 kilodaltons. In another preferred embodiment, the size exclusion chromatography is carried out using a size exclusion matrix selected from the group consisting of sephacryl 100, superdex 200 and superdex 75.

In one embodiment, the method further comprises subjecting the truncated APRIL ligand polypeptide fractions obtained following adsorption chromatography to ion exchange chromatography. Ion exchange chromatography matrices include, but are not limited to, agarose, sepharose, dextran, cross-linked cellulose and cross-linked agarose. In a preferred embodiment, the ion exchange chromatography is carried out using SP Sepharose.

Truncated APRIL Ligand Polypeptides

The invention also provides a biologically active truncated APRIL ligand polypeptide or analog thereof produced according to the methods described above. In one embodiment, the truncated APRIL ligand polypeptide or analog thereof is human. In one embodiment, the truncated human polypeptide or analog thereof is selected from the group consisting essentially of amino acid residues ranging from amino acids 115-250 of SEQ ID NO: 6 to amino acids 133-250 of SEQ ID NO: 6. In another embodiment, the truncated APRIL ligand polypeptide is a human polypeptide consisting essentially of amino acid residues 115-250 of SEQ ID NO: 6. In another embodiment, the truncated APRIL ligand polypeptide is a human polypeptide consisting essentially of amino acid residues 116-250 of SEQ ID NO: 6. In another embodiment, the truncated APRIL ligand polypeptide is a human polypeptide consisting essentially of amino acid residues 117-250 of SEQ ID NO: 6. In another embodiment, the truncated APRIL ligand polypeptide is a human polypeptide consisting essentially of amino acid residues 118-250 of SEQ ID NO: 6. In another embodiment, the truncated APRIL ligand polypeptide is a human polypeptide consisting essentially of amino acid residues 119-250 of SEQ ID NO: 6. In another embodiment, the truncated APRIL ligand polypeptide is a human polypeptide consisting essentially of amino acid residues 120-250 of SEQ ID NO: 6. In another embodiment, the truncated APRIL ligand polypeptide is a human polypeptide consisting essentially of amino acid residues 121-250 of SEQ ID NO: 6. In another embodiment, the truncated APRIL ligand polypeptide is a human polypeptide consisting essentially of amino acid residues 122-250 of SEQ ID NO: 6. In another embodiment, the truncated APRIL ligand polypeptide is a human polypeptide consisting essentially of amino acid residues 123-250 of SEQ ID NO: 6. In another embodiment, the truncated APRIL ligand polypeptide is a human polypeptide consisting essentially of amino acid residues 124-250 of SEQ ID NO: 6. In another embodiment, the truncated APRIL ligand polypeptide is a human polypeptide consisting essentially of amino acid residues 125-250 of SEQ ID NO: 6. In another embodiment, the truncated APRIL ligand polypeptide is a human polypeptide consisting essentially of amino acid residues 126-250 of SEQ ID NO: 6. In another embodiment, the truncated APRIL ligand polypeptide is a human polypeptide consisting essentially of amino acid residues 127-250 of SEQ ID NO: 6. In another embodiment, the truncated APRIL ligand polypeptide is a human polypeptide consisting essentially of amino acid residues 128-250 of SEQ ID NO: 6. In another embodiment, the truncated APRIL ligand polypeptide is a human polypeptide consisting essentially of amino acid residues 129-250 of SEQ ID NO: 6. In another embodiment, the truncated APRIL ligand polypeptide is a human polypeptide consisting essentially of amino acid residues 130-250 of SEQ ID NO: 6. In another embodiment, the truncated APRIL ligand polypeptide is a human polypeptide consisting essentially of amino acid residues 131-250 of SEQ ID NO: 6. In another embodiment, the truncated APRIL ligand polypeptide is a human polypeptide consisting essentially of amino acid residues 132-250 of SEQ ID NO: 6. In another embodiment, the truncated APRIL ligand polypeptide is a human polypeptide consisting essentially of amino acid residues 133-250 of SEQ ID NO: 6. In another embodiment, the truncated APRIL ligand polypeptide or analog thereof is murine. In another embodiment, the truncated APRIL ligand polypeptide is a murine polypeptide and consists essentially of amino acid residues 106-241 of SEQ ID NO: 5.

The invention also provides a biologically active truncated APRIL ligand polypeptide. In one embodiment, the truncated APRIL ligand polypeptide is selected from the group consisting of: (a) amino acid residues ranging from amino acids 115-250 of SEQ ID NO: 6 to amino acids 133-250 of SEQ ID NO: 6; and (b) a polypeptide encoded by a nucleotide sequence of SEQ ID NO: 7. In another embodiment, the truncated APRIL ligand polypeptide is selected from the group consisting of: (a) amino acid residues 106-241 of SEQ ID NO: 5; and (b) a polypeptide encoded by a nucleotide sequence of SEQ ID NO: 1. In some embodiments, the truncated APRIL ligand polypeptide consists essentially of a polypeptide encoded by a nucleotide sequence of SEQ ID NO: 7. In another embodiment, the truncated APRIL ligand polypeptide consists essentially of a polypeptide encoded by a nucleotide sequence of SEQ ID NO: 1.

The present invention also includes analogs of the N-terminal truncated APRIL ligand polypeptides encoded by amino acid residues ranging from amino acids 115-250 of SEQ ID NO: 6 to amino acids 133-250 of SEQ ID NO: 6 or polypeptides encoded by amino acid residues 106-241 of SEQ ID NO: 5 described herein, that result from further truncations and/or amino-acid substitutions to the truncated APRIL ligand polypeptide. Similarly, the invention also includes analogs of the truncated APRIL ligand polypeptides disclosed herein that may result from in vivo or in vitro chemical derivatization. Such derivatization includes, but is not limited to, changes in acetylation, methylation, phosphorylation, carboxylation, oxidation state, or glycosylation. In addition, chemical derivatization may involve coupling to organic polymers such as polyethylene glycol (PEG) or other polymers known in the medicinal arts. Thus, a truncated APRIL ligand polypeptide analog may result from a non-amino acid sequence modification. Therefore, whether the truncated APRIL ligand polypeptide is expressed for the purposes of retaining wild-type activity, a modified activity, or as an antagonist of APRIL ligand/receptor activity, the invention provides truncated APRIL ligand polypeptide analogs having this critical N-terminal truncation, as well as methods for expressing and purifying the analogs.

The truncated APRIL ligand polypeptides disclosed herein may be expressed as fusion proteins. Fusion proteins are well known in the art. A person of skill in the art may choose from a wide variety of fusion partner moieties, including those from prokaryotes and eukaryotes. (See, e.g., Molecular Cloning A Laboratory Manual, 2^{nd} Ed., ed. by Sambrook et al. (Cold Spring Harbor Laboratory Press 1989) and Current Protocols in Molecular Biology, ed. by Ausubel et al. (Greene Publishing and Wiley Interscience, New York 1998), the contents of which are herein incorporated by reference). In one embodiment, the fusion partner moieties include, but are not limited to, toxic peptide moieties, complement proteins, radiolabeled proteins, cytokines, or antibiotic proteins and immunoglobulin fragments. In one embodiment, the fusion partner moiety is a Myc tag or a FLAG tag. Fusion partner moieties may be of animal (including mammal, e.g., human), bacterial, yeast, insect, or viral origin.

Nucleic Acids Encoding Truncated APRIL Ligand Polypeptides

The invention also provides isolated nucleic acid molecules comprising a nucleotide sequences encoding the biologically active truncated APRIL ligand polypeptides or analogs thereof of the present invention. In some embodiments, the nucleic acid molecule is human. In some embodiments, the nucleotide sequence encoding a truncated APRIL ligand polypeptide is SEQ ID NO: 7. In some embodiments, the nucleic acid molecule is murine. In some embodiments, the nucleotide sequence encoding a truncated APRIL ligand polypeptide is SEQ ID NO: 1. In some embodiments, the nucleic acid molecule encodes a truncated APRIL ligand polypeptide fused to a fusion partner. In some embodiments, the fusion partner is a Myc tag. In some embodiments, the fusion partner is a FLAG tag.

Pharmaceutical Compositions

The biologically active truncated APRIL ligand polypeptides, analogs thereof and fusion proteins disclosed herein may be formulated in pharmaceutical compositions by the methods disclosed herein and may be delivered by a parenteral route, injection, transmucosal, oral, inhalation, ocular, rectal, long-acting implantation, topical, sustained-released or stent-coated means. APRIL ligand polypeptides may be in a variety of conventional forms employed for administration. These include, for example, solid, semi-solid and liquid dosage forms, such as liquid solutions or suspension, slurries, gels, creams, balms, emulsions, lotions, powders, sprays, foams, pastes, ointments, salves, and drops.

The most effective mode of administration and dosage regimen of the biologically active truncated APRIL ligand polypeptides, analogs thereof and fusion proteins, or compositions comprising them, will depend on the effect desired, previous therapy, if any, the individual's health status, the status of the condition itself, the response to the truncated APRIL ligand polypeptides, analogs thereof and fusion proteins, and the judgment of the treating physician. Truncated APRIL ligand polypeptides, analogs thereof and fusion proteins, or compositions comprising them, may be administered in any dosage form acceptable for pharmaceuticals or veterinary preparations, at one time or over a series of treatments.

The amount of truncated APRIL ligand polypeptides, analogs thereof and fusion proteins, or compositions comprising them, which provides a single dosage, will vary depending upon the particular mode of administration, the specific APRIL ligand polypeptide, analog thereof and fusion protein or composition, dose level, and dose frequency. A typical preparation will contain between about 0.01% and about 99%, preferably between about 1% and about 50%, of an APRIL ligand polypeptide or compositions thereof (w/w).

An exemplary, non-limiting range for a therapeutically or prophylactically effective amount of a truncated APRIL ligand polypeptide, analog thereof or fusion protein is between about 0.005-10.00 mg/kg body weight, more preferably between about 0.05-1.0 mg/kg body weight.

Truncated APRIL ligand polypeptides, analogs thereof or fusion proteins, or compositions comprising them, may be administered alone, or as part of a pharmaceutical or veterinary preparation, or as part of a prophylactic preparation, with or without adjuvant. They may be administered by parenteral, oral, pulmonary, nasal, aural, anal, dermal, ocular, intravenous, intramuscular, intraarterial, intraperitoneal, mucosal, sublingual, subcutaneous, transdermal, topical or intracranial routes, or into the buccal cavity. In either pharmaceutical or veterinary applications, truncated APRIL ligand polypeptides, analogs thereof or fusion proteins may be topically administered to any epithelial surface. Such surfaces include oral, ocular, aural, anal and nasal surfaces.

Pharmaceutical compositions for use in accordance with the present invention may be formulated in any conventional manner, using one or more physiologically acceptable carriers comprising excipients and auxiliaries which facilitate processing of the active compounds into preparations which can be used pharmaceutically. The appropriate formulation will be dependent upon the intended route of administration. Pharmaceutical compositions may be produced by means of conventional mixing, dissolving, granulating, dragee-making, levigating, emulsifying, encapsulating, entrapping or lyophilizing processes.

For transmucosal administration, penetrants appropriate to the barrier to be permeated are used in the formulation. Such penetrants are generally known in the art. For ocular administration, suspensions in an appropriate saline solution are used, as is known in the art.

For oral administration, the truncated APRIL ligand polypeptides, analogs thereof and fusion proteins may be formulated readily by combining the active agents with conventional pharmaceutically acceptable carriers. Truncated APRIL ligand polypeptides, analogs thereof and fusion proteins may be formulated as tablets, pills, liposomes, granules, spheres, dragees, capsules, liquids, gels, syrups, slurries, suspensions and the like, for oral ingestion by a patient to be treated.

The invention provides a pharmaceutical composition comprising a truncated APRIL ligand polypeptide, analog thereof or fusion protein and a therapeutically acceptable carrier, adjuvant or vehicle. These carriers and adjuvants and vehicles include, for example, Freund's adjuvant, ion exchanges, alumina, aluminum stearate, lecithin, buffer substances, such as phosphates, glycine, sorbic acid, potassium sorbate, partial glyceride mixtures of saturated vegetable fatty acids, waters, salts or electrolytes, such as protamine sulfate, disodium hydrogen phosphate, sodium chloride, zinc salts, colloidal silica, magnesium, trisilicate, cellulose-based substances and polyethylene glycol. Adjuvants for topical or gel base forms may include, for example, sodium carboxymethylcellulose, polyacrylates, polyoxyethylene-polyoxypropylene-block polymers, polyethylene glycol and wood wax alcohols.

Pharmaceutical compositions for oral use can be obtained as a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All compositions for oral administration should be in dosages suitable for such administration.

For buccal administration, the compositions may take the form of tablets or lozenges formulated in a conventional manner. For administration by inhalation, truncated APRIL ligand polypeptides, analogs thereof or fusion proteins are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

Truncated APRIL ligand polypeptides, analogs thereof and fusion proteins may be formulated for either parenteral administration by injection, e.g., by bolus injection, or continuous infusion. The agents may be formulated in aqueous solutions, aqueous suspensions, oily suspensions, or emulsions, and may contain formulatory agents such as suspending, stabilizing and/or dispersing agents. Formulations for injection may be presented in unit dosage form, e.g., in ampoules or in multi-dose containers, with an added preservative.

Typical aqueous solution formulations include physiologically compatible buffers such as Hank's solution, Ringer's solution, or physiological saline buffer. Typical oily suspensions may include lipophilic solvents or vehicles that include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspensions may also contain suitable stabilizers or agents which increase the solubility of the compounds to allow for the preparation of highly concentrated solutions. Alternatively, truncated APRIL ligand polypeptides, analogs thereof and fusion proteins may be in powder form for constitution with a suitable vehicle, such as sterile pyrogen-free water, before use.

The truncated APRIL ligand polypeptides, analogs thereof and fusion proteins may also be formulated in rectal compositions, such as suppositories or retention enemas, e.g., containing conventional suppository bases such as cocoa butter or other glycerides.

In addition to the formulations described, truncated APRIL ligand polypeptides, analogs thereof and fusion proteins may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for truncated APRIL ligand polypeptides, analogs thereof and fusion proteins which are hydrophobic is a co-solvent system comprising benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may be substituted for dextrose.

Alternatively, other delivery systems for hydrophobic pharmaceutical compounds may be employed. Liposomes and emulsions are examples of delivery vehicles or carriers for hydrophobic drugs. Certain organic solvents, such as dimethylsulfoxide also may be employed, although they may display a greater toxicity.

Additionally, truncated APRIL ligand polypeptides, analogs thereof and fusion proteins may be delivered using a sustained-release system, such as semipermeable matrices of solid hydrophobic polymers containing the therapeutic agent. Various sustained-release materials are available and well known by those skilled in the art. Sustained-release capsules may, depending on their chemical nature, release the compounds for a few weeks up to over 100 days.

Depending on the chemical nature and the biological stability of the truncated APRIL ligand polypeptides, analogs thereof or fusion proteins, additional strategies for protein stabilization may be employed.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Truncated APRIL ligand polypeptides, analogs thereof and fusion proteins may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Truncated APRIL ligand polypeptides, analogs thereof and fusion proteins may also be formulated into pharmaceutical compositions useful for coating stents.

Methods of Using Truncated APRIL Ligand Polypeptides

The widespread expression of APRIL ligand in tumor cells and lymphoid cells suggests an important role for APRIL ligand in tumorigenesis and diseases of the immune system, e.g., immunodeficiencies. Also, because of the role APRIL ligand has in tumor cell proliferation, modified or inhibitory APRIL ligand polypeptides may form the basis of effective treatments for cancer.

The truncated APRIL ligand polypeptides, analogs thereof and fusion proteins of this invention may be used to induce or promote cell proliferation in an immunosuppressed subject comprising administering to a subject a therapeutically effective amount of a truncated APRIL ligand polypeptide, analog thereof, fusion protein or pharmaceutical composition of this invention. It may be useful to induce or promote proliferation of cells such as immune cells (e.g., T cells, B cells, macrophages, neutrophils, eosinophils, basophils, mast cells), for example, in treating immunosuppressed subjects lacking the normal repertoire of cellular populations.

The truncated APRIL ligand polypeptide fusion proteins and analogs thereof of this invention may be used to treat or reduce the severity of conditions, mediated at least in part, by APRIL ligand in a subject, comprising administering a fusion protein comprising a truncated APRIL ligand polypeptide disclosed herein and a toxic fusion partner moiety. The truncated APRIL ligand polypeptide portion of the fusion protein is used to target the fusion protein to tissues expressing the APRIL receptor (BCMA and TACI) whereas the toxic fusion partner portion delivers a toxin to the target cell. The methods disclosed herein may be useful in treating or reducing the severity of conditions, for example, of epithelial and fibroblastic tumor cells, of T and B cell lymphoma and leukemic cells, and of peripheral blood mononuclear cells, such as peripheral blood B cells.

The truncated APRIL ligand polypeptide fusion proteins and analogs thereof of this invention may also be used to treat or reduce an inflammatory response, mediated at least in part, by APRIL ligand in a subject, comprising administering a fusion protein comprising a purified truncated APRIL ligand polypeptide disclosed herein and a toxic fusion partner moiety. The truncated APRIL ligand polypeptide portion of the fusion protein is used to target the fusion protein to tissues expressing the APRIL receptor (BCMA and TACI) whereas the toxic fusion partner portion delivers a toxin to the target cell. The methods disclosed herein may be useful in treating or reducing an inflammatory response, for example, from human rheumatoid arthritis synovial tissue, cells isolated from the blood of autoimmune patients (e.g., lupus patients) and cells isolated from other chronic inflammatory sites including diseased periodontal tissue, atopic dermatis or psoriatic plaques, inflamed colon, prostate, or other target tissues.

In one embodiment, the methods of treatment according to the present invention further comprise the step of administering to the subject at least one additional agent. In another embodiment, the additional agent is selected from the group consisting of IFN-γ, IL-1B and TNF. Alternatively, such an additional agent may be co-administered to the subject with a truncated APRIL ligand polypeptide, analog thereof or fusion protein of this invention.

### EXAMPLES

In order that this invention may be better understood, the following examples are set forth. These examples are for the purpose of illustration only and are not to be construed as limiting the scope of the invention in any manner.

### Example 1

### Truncated Murine APRIL Ligand Polypeptide Expression

Peptide sequence analysis of murine APRIL ligand polypeptide secreted by 293T cells transfected with full length APRIL ligand cDNA has revealed that APRIL ligand polypeptide is cleaved between residues R95 and A96 (data not shown). Thus, a mature, secreted form of the murine APRIL ligand polypeptide begins at amino acid 96.

Truncated murine APRIL ligand DNA was amplified from an expressed sequence tag clone (obtained from the Image Consortium) using the 5′ primer disclosed in SEQ ID NO: 2 and the 3′ primer disclosed in SEQ ID NO: 3. A DNA cassette (SEQ ID NO: 1) encoding a truncated murine APRIL ligand polypeptide sequence (amino acids 106-241, i.e., SEQ ID NO: 4) downstream of a Sac1 site was used to clone into a pIC9-derived vector (Invitrogen, Purchase, N.Y.) containing a myc-tag, a glycine(×4)/serine linker, and a KEL sequence (from Fas-L, which creates a Sac1 site). A vector derived Not1 site was used at the 3′ end. The truncated murine APRIL ligand polypeptide under the control of a methanol oxidase promoter (Research Corporation Technologies, 101 North Wilmot Road, Suite 600 Tucson, Ariz.) was expressed in yeast (Pichia pastoris strain GS115) grown in BMMY medium (Invitrogen, Purchase, N.Y.) according to the method described in Rennert et al. J. Exp. Med. 192:1677-1684 (2000).

The DNA sequence set forth in SEQ ID NO: 1, expresses the truncated murine APRIL ligand polypeptide extracellular domain from amino acids 106-241 (SEQ ID NO: 4), a fragment that lacks the lysine rich region but retains the functional domains that are conserved across the other TNF family ligands (BAFF, TWEAK, EDA).

### Example 2

### Truncated Murine APRIL Ligand Polypeptide Purification

To obtain highly purified murine APRIL ligand polypeptide from the Pichia pastoris cultures prepared in Example 1, the media was concentrated five fold and dialyzed with 20 mM sodium phosphate (pH 7.0) using a mini Pellicon II apparatus (Millipore Corp. Bedford, Mass.), with a Biomax 10 filter (10 kDa molecular weight cutoff (MWCO), Millipore Corp., Bedford, Mass.) until the conductivity of the material reached approximately 3.5 millisiemens per square centimeter (mS/cm²).

The dialysed concentrate was loaded onto a 10 mL hydroxyapatite column (CHT-10, Biorad, Hercules, Calif.) and washed with 5 column volumes of starting buffer (20 mM sodium phosphate pH 7.0). The column was then eluted with a linear gradient from 20 to 200 mM sodium phosphate pH 7.0, over 10 column volumes. The eluted fractions were analyzed for purity by SDS PAGE and fractions containing murine APRIL ligand polypeptide were pooled.

The polypeptide pool was then concentrated approximately 10-fold using a Biomax spin concentrator (10 kDa MWCO). Gel filtration of the concentrate was achieved using either a Sephacryl 100 high-resolution (HR) column (2.6 cm diameter x 100 cm length), or a Superdex 75 column (Pharmacia Corp., Piscataway, N.J.) to separate higher molecular weight contaminants from truncated murine APRIL ligand polypeptide aggregates. Two major peaks eluted from the column, with the later eluting peak containing truncated murine APRIL ligand polypeptide.

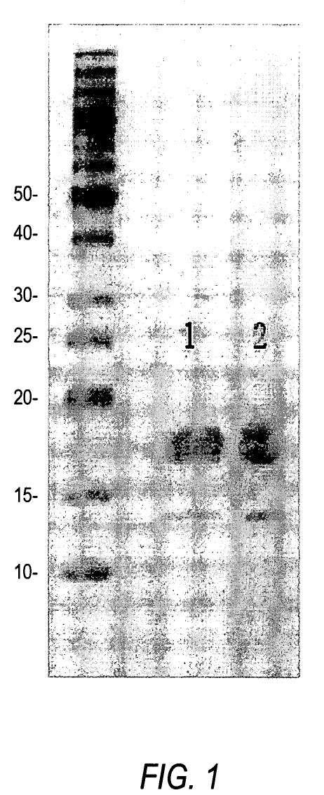

The fractions were analyzed for purity by SDS PAGE; fractions containing greater than 90% pure APRIL ligand polypeptide were pooled. The truncated APRIL ligand polypeptide has a molecular weight of approximately 17 kDa, as shown by SDS-PAGE electrophoresis (FIG. 1), with two minor bands observed at 15 and 10 kDa.

The truncated murine APRIL ligand polypeptide encoded by the yeast expression vector was expressed with a myc tag, glycine linker, and an amino acid sequence encoded by the nucleotide linker, on the N-terminus. However, during the Pichia pastoris fermentation, endogenous proteases cleaved between 95-100% of the myc tag between E8 and D9 of the myc sequence but the linker sequences remained intact, as did the entire truncated APRIL ligand polypeptide sequence from amino acids 106-241 of SEQ ID NO: 5. No uncleaved species were observed within the limits of detection. Thus, an innocuous, short amino acid sequence remained on the N-terminus of the soluble APRIL ligand. FIG. 1 shows reducing and nonreducing PAGE of the purified myc-tagged truncated murine APRIL ligand polypeptide.

The 17 kDa protein was confirmed to be an APRIL ligand polypeptide by western analysis using a rat anti-murine APRIL ligand polypeptide IgG2b antibody. Endotoxin analysis was carried out using a Polychrome LAL Endotoxin Kit (Associates of Cape Cod, Woods Hole, Mass.), and was measured as 0.6 endotoxin units per milligram (EU/mg).

Mass spectrometric analysis was done on the myc-tagged truncated murine APRIL ligand polypeptide purified over an SDS trap connected in series with a C4 guard column and analyzed on-line by electrospray ionization mass spectrometry (ESI-MS) using a triple quadrupole instrument (Micromass Quattro II, Beverly, Mass.). The raw data was deconvoluted using the MaxEnt program.

Mass spectrometry (1) showed the molecular weight of the expressed truncated APRIL ligand polypeptide to be 16,237 daltons, which agrees with the approximate 17 kDa molecular weight predicted by the SDS-PAGE analysis, (2) confirmed the N-terminal sequencing which showed the proteolytic removal of the myc tag, (3) confirmed that the C-terminus of the truncated APRIL ligand polypeptide remained intact, and (4) showed some amount of protein O-glycosylation.

The typical yield from 1 liter of Pichia pastoris fermentation broth was approximately 9.6 mg of purified truncated APRIL ligand polypeptide, with greater than 90% purity as assessed by SDS PAGE, and low endotoxin activity (<1EU/mL).

### Example 3

### Truncated Human APRIL Ligand Expression

Human APRIL ligand DNA encoding a polypeptide consisting of amino acid residues 115-250 of the human APRIL full-length sequence (SEQ ID NO: 6), was amplified from the PL449 plasmid using the 5′ primer disclosed in SEQ ID NO: 9 and the 3′ primer disclosed in SEQ ID NO: 10. A DNA cassette (SEQ ID NO: 7) encoding a truncated human APRIL ligand polypeptide sequence (amino acids 115-250 of SEQ ID NO: 6) downstream of a Pst-1 site was used to clone into a PCRIII vector (Invitrogen, Carlsbad, Calif.) containing a hemaglutinin signal for protein secretion in eukaryotic cells and a N-terminal FLAG epitope. A vector derived Not-I site was used at the 3′ end.

Truncated human FLAG-tagged APRIL ligand polypeptides having N-terminal truncations were then transiently transfected into human kidney 293T cells using the lipofectamine method of transfection. Briefly, the cells were grown to 70% confluency in DMEM media prior to transfection with Lipofectamine 2000 (Invitrogen, Carlsbad, Calif.). Cells were then maintained post-transfection in serum-free DMEM media and the conditioned media harvested on days 3 and 6. Harvested media was filter sterilized, aliquoted and frozen until further characterization and purification.

### Example 4

### Truncated Human APRIL Ligand Polypeptide Purification

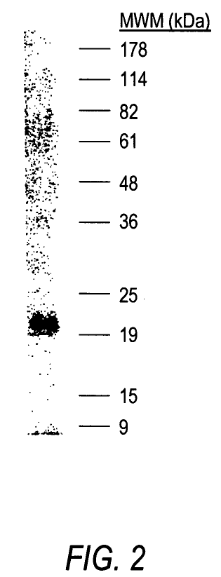

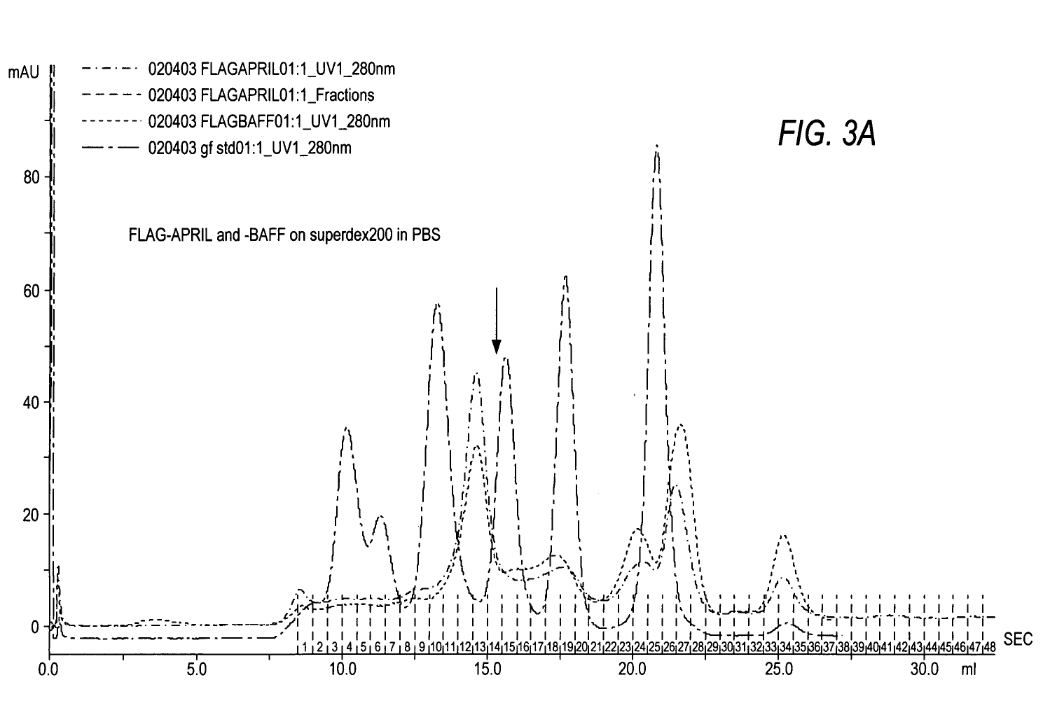

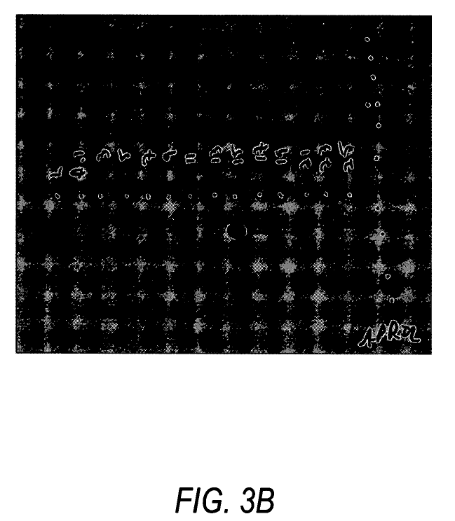

In order to obtain highly purified truncated human APRIL ligand polypeptide from human kidney 293T cell cultures expressing FLAG-tagged truncated human APRIL ligand polypeptide, the conditioned media was concentrated to a final concentration of 10 mM CaCl₂ and 150 mM NaCl before filtering through a 0.2 μm filter. The medium was loaded onto an anti-FLAG M1 Separose affinity column (A4596, Sigma-Aldrich Corp., St. Louis, Mo.) three times by gravity at 4° C. The anti-FLAG M1 Sepharose column was used to purify N-terminal FLAG fusion proteins by utilizing a monoclonal antibody that binds to the FLAG protein. The column was than washed with 10 column volumns of buffer (50 mM Tris pH 7.6, 150 mM NaCl, 1 mM CaCl₂). The FLAG-tagged truncated human APRIL ligand polypeptide was gently eluted from the column with 5 column volumns of buffer (50 mM Tris pH 7.6, 150 mM NaCl) containing 100 μg/mL FLAG peptide (F3290, Sigma-Aldrich Corp., St. Louis, Mo.). The eluted fractions were analyzed for purity by SDS-PAGE and fractions containing truncated human APRIL ligand polypeptide were dialyzed overnight into 20 mM Tris pH 6.8 using a Slide-a-lyzer Dialysis cassette with a 10 kDa molecular weight cutoff (MWCO) (Pierce, Rockford, Ill.). The dialyzed pool was then loaded onto a HiTrap™ SP column (SP Sepharose)(Pharmacia, Piscataway, N.J.) and the column was washed with four column volumes of 20 mM Tris pH 6.8. The HiTrap™ SP column utilizes a cation ion exchange media based on SP Sepharose for purifying proteins. FLAG-tagged truncated human APRIL ligand polypeptide was eluted from the column in a single step with 1M NaCl in 20 mM Tris pH 6.8. The eluted fractions containing protein, as identified using SDS-PAGE, was subjected to a further purification step using a gel filtration column (Superdex 200 (10 mm×300 cm)) (Pharmacia, Piscataway, N.J.). Analysis by non-reducing SDS-PAGE revealed two closely spaced bands of 21 kDa and 22 kDa (FIG. 2), most likely representing glycosylated and non-glycosylated forms of the protein.

Purified FLAG-tagged truncated human APRIL ligand polypeptide was run on an analytical gel filtration column (Superdex 200 (10 mm×30 cm)) and eluted as a 60 kDa trimeric protein, based on comparison with molecular size standards. This suggests that the truncated human APRIL ligand polypeptide produced by the methods described herein, is biologically active because other TNF family ligands are also known to arrange in a trimeric configuration in order to exhibit biological activity.

### Example 5

### Longer Forms of Soluble Human APRIL Ligand Polypeptide Contain High Molecular Weight Aggregates

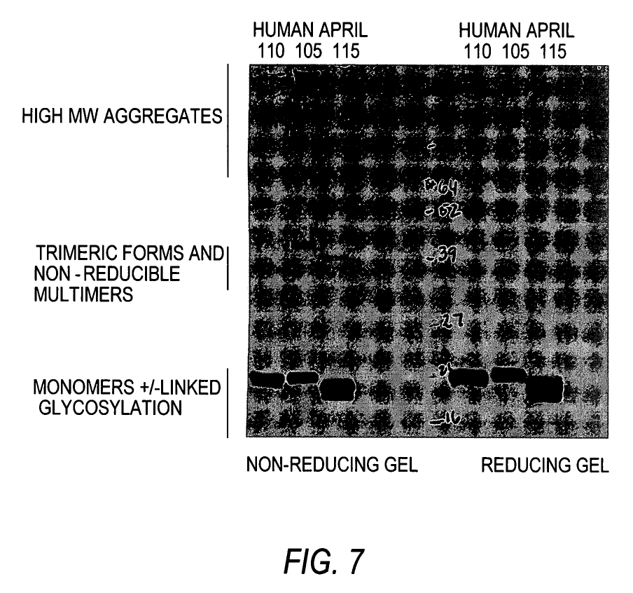

A major problem with protein expression and purification systems involves insoluble protein aggregation. Full-length APRIL ligand polypeptides contain an N-terminal lysine-rich region that leads to protein aggregation when expressed at high levels. Various truncated forms of the human APRIL ligand polypeptide lacking the N-terminal lysine-rich region are disclosed herein. These different truncated forms of the human APRIL ligand polypeptide consisting of amino acids 105-250 of SEQ ID NO: 6, 110-250 of SEQ ID NO: 6 and 115-250 of SEQ ID NO: 6 contained varying amounts of high molecular aggregates when analyzed using SDS-PAGE at a 4-20% gradient. Under non-reducing conditions, SDS-PAGE analysis showed that only the shortest truncated human APRIL ligand polypeptide encoded by amino acid residues 115-250 of SEQ ID NO: 6 was devoid of any high molecular weight aggregates. In contrast, the longer forms of the truncated human APRIL ligand polypeptide (i.e., encoded by amino acid residues 105-250 of SEQ ID NO: 6 or 110-250 of SEQ ID NO:6) contained high molecular weight aggregates (see FIG. 7). Under reducing conditions, only the monomeric and non-reducible forms of the molecules were observed (see FIG. 7). Thus, SDS-PAGE analysis under reducing conditions eliminated the aggregates observed under non-reducing conditions. SDS-PAGE analysis permits the separation of multimeric proteins into individual, mostly linear polypeptide chains (i.e., without higher-order structure) that migrate in the gel according to relative size in a manner that corresponds closely with their relative mass or molecular weight. Thus, the truncated APRIL ligand polypeptide encoded by amino acids residues 115-250 of SEQ ID NO: 6 was able to prevent and overcome the aggregation problems previously encountered with full-length APRIL ligand polypeptides.

In addition, the shortest truncate of APRIL ligand polypeptides (amino acid residues 115-250 of SEQ ID NO: 6) was detectable on SDS-PAGE as a trimeric molecule under non-reducing conditions (see FIG. 7). Thus, the truncated human APRIL ligand polypeptide encoded by amino acids residues 115-250 of SEQ ID NO: 6 was not only devoid of aggregates but also existed as a biologically active trimeric molecule.

It may be possible to further truncate the N-terminal region of the human APRIL ligand polypeptide using the methods described herein, wherein such further truncations results in APRIL ligand polypeptides encoded by amino acid residues ranging from amino acids 116-250 of SEQ ID NO: 6 to amino acids 133-250 of SEQ ID NO: 6. Such further N-terminal truncations will result in the removal of amino acids which are predicted to not significantly affect the overall charge of the protein. Such further N-terminal truncations can be efficiently purified using the methods described herein to yield biologically active truncated human APRIL ligand polypeptides with the property to trimerize.

### Example 6

### Purified Truncated Murine APRIL Ligand Polypeptide Specifically Binds to APRIL Receptors

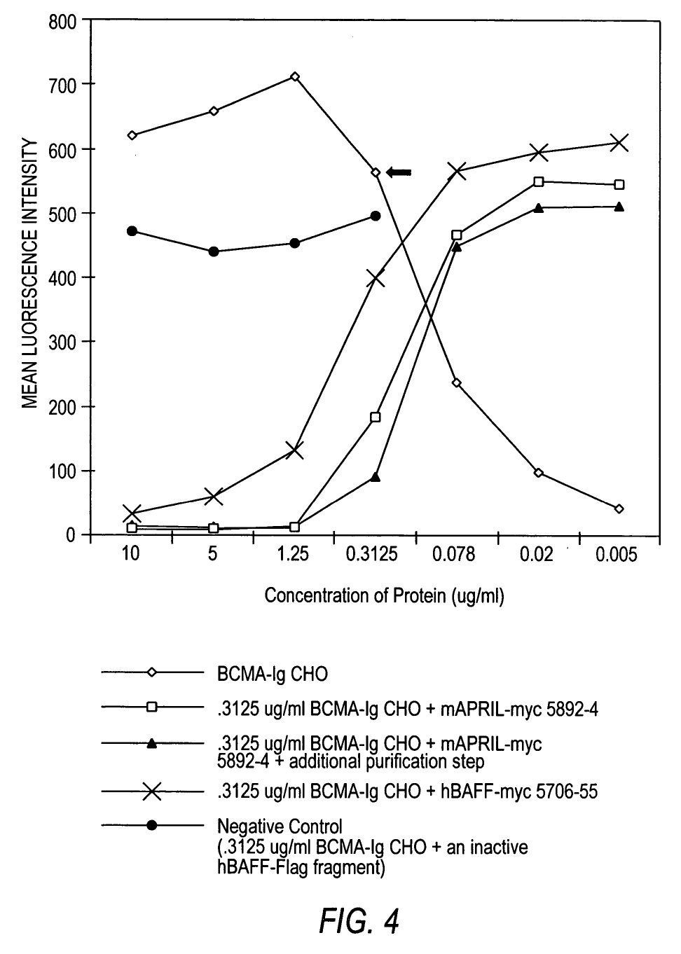

Assays for affinity binding of truncated murine APRIL ligand to BCMA and TACI receptor-expressing cell lines showed that the truncated murine APRIL ligand polypeptide (amino acids 106-241 of SEQ ID NO: 5) purified from P. pastoris binds these receptors with high affinity (see FIG. 4).

When different concentrations of truncated murine APRIL ligand polypeptide ranging from 0 to 10 μg/mL were incubated with 0.3125 μg/mL of BCMA-Ig protein and 5×10⁵ APRIL ligand-expressing 293T cells, truncated murine APRIL ligand polypeptides (amino acids 106-241 of SEQ ID NO: 6) produced using the methods disclosed herein were able to efficiently compete with the cell surface APRIL ligand for binding to the BCMA-Ig molecule as the concentration of truncated murine APRIL ligand polypeptide was increased (see FIG. 4).

Fluorescence activated cell sorting (FACS) analyses demonstrate that truncated murine APRIL ligand polypeptides (amino acids 106-241 of SEQ ID NO: 5) produced using the method disclosed herein were able to efficiently compete with the cell surface APRIL ligand for binding to the BCMA-Ig molecule. This was demonstrated by the decreased binding of the BCMA-Ig molecule to cell surface APRIL ligand as the concentration of soluble APRIL ligand was increased. To ensure that the binding observed was specific, an inactive hBAFF-FLAG molecule was used as a negative control and was unable to compete with cell surface APRIL ligand for binding to the BCMA-Ig molecule (see FIG. 4).

Thus, the truncated murine APRIL ligand fragment of SEQ ID NO: 4 (i.e., amino acids 106-241 of SEQ ID NO: 5) that was expressed in Pichia pastoris and isolated to a high degree of purity binds specifically to APRIL receptors.

### Example 7

### Purified Truncated Human APRIL Ligand Polypeptide Specifically Binds to APRIL Receptors

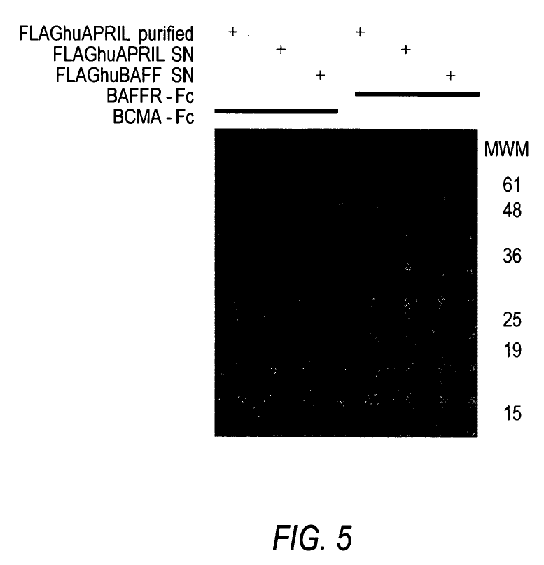

Purified FLAG-tagged truncated human APRIL ligand polypeptides produced by the methods disclosed herein binds specifically to its receptor with high affinity (see FIG. 5). The ability of APRIL ligand polypeptide to selectively bind to its receptors (BCMA and TACI) was used to determine if the truncated human APRIL ligand polypeptide retained its proper binding specificity. A related TNF family ligand, BAFF, has been found to also bind to TACI and BCMA, in addition to binding to the BAFF receptor (BAFFR), which does not bind the APRIL ligand polypeptide. Thus, IgG1 Fc protein fusions of the two receptors, BCMA-Fc and BAFFR-Fc, were generated. These receptor fusion proteins can still bind to their respective ligands and bind to Protein A coated beads through the Fc portion of the protein.

Purified FLAG-tagged truncated human APRIL ligand polypeptide (1 μg, FLAGhuAPRIL purified), unpurified FLAG-tagged truncated human APRIL ligand polypeptide in cell medium (1 μg, FLAGhuAPRIL SN) and unpurified FLAG-tagged human BAFF ligand polypeptide in cell medium (1 μg, FLAGhuBAFF SN) was added to 1 ml of 2% fetal bovine serum in PBS containing 10 μl of Protein A beads in the presence of 1 μg of BCMA-Fc or BAFFR-Fc. The binding reaction was incubated at 4° C. overnight. The beads were spun down and the supernatant was removed. The beads were resuspended in 1 mL FBS/PBS buffer and transferred to a tube. The beads were again spun down and the supernatant was removed. The beads were then resuspended in 10 μl of 2×SDS-PAGE reducing buffer and the samples were run on SDS-PAGE and transferred onto nitrocellulose. These western blots were probed with anti-FLAG M2 antibody (Sigma-Aldrich Corp, St. Louis, Mo.). FLAG-tagged truncated human APRIL ligand polypeptide, both purified and unpurified, bound specifically to BCMA and not to BAFFR. BAFF was found to bind to both BCMA and BAFFR. Therefore, the FLAG-tagged truncated human APRIL ligand polypeptide produced by the methods herein retains its receptor specificity following purification.

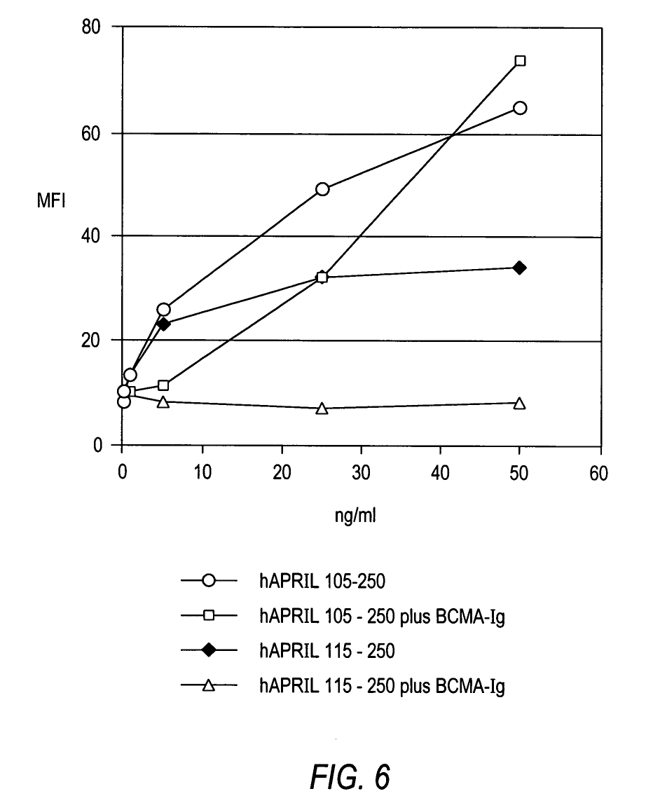

Further binding studies using truncated human APRIL ligand polypeptide to a TACI+ expressing IM9 cell line (ATCC, Rockville Md.) demonstrated that while both the longer (amino acid residues 105-250 of SEQ ID NO: 6) and shorter (amino acid residues 115-250 of SEQ ID NO: 6) truncates of human APRIL ligand polypeptide were capable of binding, only the shorter truncate (amino acid residues 115-250 of SEQ ID NO: 6) of the human APRIL ligand polypeptide could be specifically competed off by the soluble BCMA-Ig fusion protein (see FIG. 6). This result showed that a major component of the binding of the longer truncate (amino acid residues 105-250 of SEQ ID NO: 6) of human APRIL ligand polypeptide to TACI+IM9 cells was nonspecific.

Increasing concentrations, ranging from 1 to 50 ng/mL, of either truncated human APRIL ligand polypeptide encoded by amino acid residues 105-250 of SEQ ID NO: 6 or amino acid residues 115-250 of SEQ ID NO: 6, prepared as described in Examples 3 and 4, were incubated with TACI+IM9 cells in the presence or absence of BCMA-Ig protein and the binding was detected using fluorescent activated cell sorting (FACS).

### Example 8

### Purified Truncated Murine APRIL Ligand Polypeptide Retains a High Level of Biological Activity

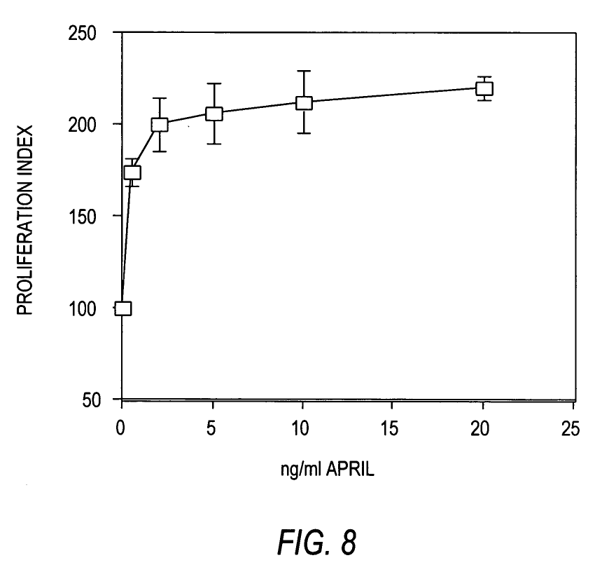

Exposing tumor cells to APRIL ligand has been previously shown to enhance tumor cell growth. See Ware J. Exp. Med. 192:F35-38 (2000). These results were expanded upon with a truncated murine APRIL ligand polypeptide made using the methods disclosed herein. The growth of APRIL ligand-responsive cells in low serum cultures was enhanced by the addition of the truncated murine APRIL ligand polypeptides (amino acids 106-241 of SEQ ID NO: 5). As little as 1 ng/ml of the trimeric recombinant murine APRIL ligand polypeptide shown in FIG. 8 (less than 1 pM) was sufficient to induce NIH-3T3 cell proliferation. At 5 ng/ml, cell growth had reached a plateau, demonstrating that the truncated murine APRIL ligand polypeptides disclosed herein retain a high level of biological activity.

### Example 9

### The Truncated Murine APRIL Ligand Polypeptide Acts in Concert with Known Growth Factors

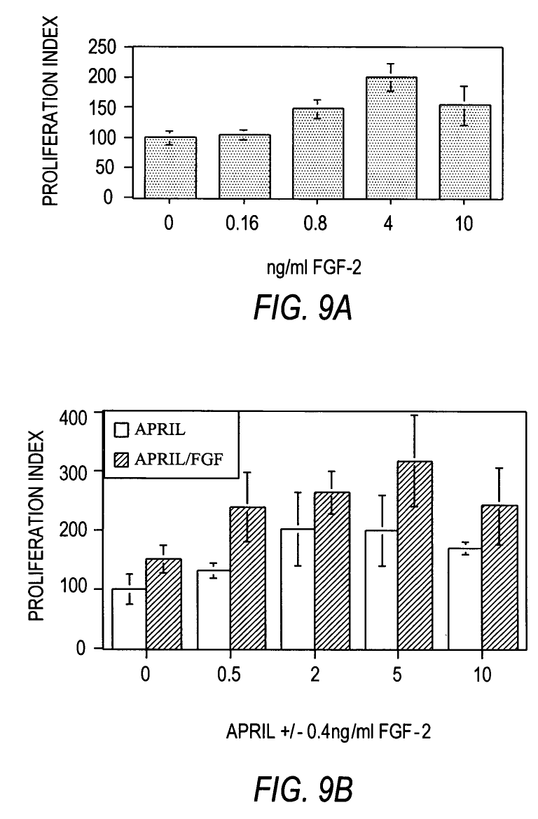

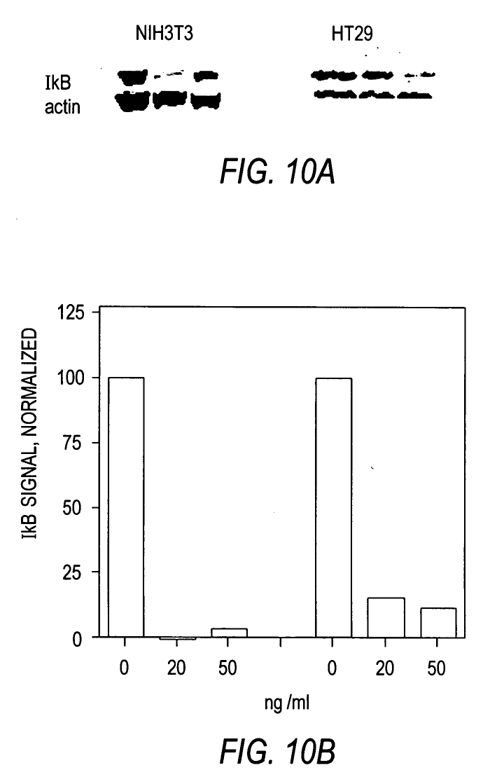

NIH-3T3 cells were plated at 2.5×10³ cells on 96-well plates and serum-starved for 16 h. Cells were cultured in 1% FBS with increasing amounts of FGF-2 (see FIG. 9A). A dose response curve for FGF-2-induced proliferation is shown in FIG. 9A, as measured by ³H-thymidine incorporation. Cultures containing less than 1 ng/ml of FGF-2 alone showed a suboptimal response. In contrast, cells cultured in 1% FBS containing 0.4 ng/ml of FGF-2 with increasing concentrations of truncated murine APRIL ligand polypeptide (amino acids 106-241 of SEQ ID NO: 5) made using the methods disclosed herein, showed dramatic increases in cellular proliferation with the addition of as little as 0.5 ng/ml of a truncated murine APRIL ligand polypeptide (see FIG. 9B). Cells were cultured for 3 days, and graphs are shown as the percent increase in [³H]-thymidine incorporation above control.

Upon increasing the concentration of APRIL ligand, while maintaining a constant concentration of FGF-2, cellular proliferation increased by as much as 300% over untreated cell cultures, reaching maximal proliferation at 5 ng/ml. Each bar represents the mean value derived from triplicate cell cultures and the error bars show ±1 standard error of measurement (SEM).

### Example 10

### Truncated Trimeric APRIL Ligand Polypeptide-Induced Signaling

APRIL ligand polypeptide has previously been shown to enhance tumor cell growth. See Ware J. Exp. Med. 192:F35-38 (2000). The underlying signaling mechanism by which APRIL ligand polypeptide is involved in the regulation of tumor cell growth was demonstrated using the truncated human and murine APRIL ligand polypeptides produced using the methods disclosed herein.

Nuclear factor-kappa B (NF-κB) is a highly inducible transcription factor that participates in diverse biological processes, including innate/adaptive immunity and cellular survival through the induction of genetic networks. The major transcriptional-activating species Rel A-NF-κB is a cytoplasmic complex whose nuclear translocation is controlled by its association with a family of inhibitory proteins, termed IkappaBs (IκB). Activation of the NF-κB pathway results as a consequence of the targeted proteolysis of IκB, releasing NF-κB to enter the nucleus and bind to specific sequences in target promoters, such as those involved in protecting the cell from undergoing apoptosis.

Both truncated murine (amino acid residues 106-241 of SEQ ID NO: 5) and human (amino acid residues 115-250 of SEQ ID NO: 6) APRIL ligand polypeptides produced by the methods disclosed herein were able to activate the NF-κB signaling pathway in tumor cells. This was demonstrated by a decrease in IκB levels in the presence of increasing concentrations of truncated APRIL ligand polypeptide.

NIH-3T3 cells or HT29 cells were grown to 70% confluency in 6-well plates and then serum starved in low serum for 24 h in DMEM media. NIH3T3 cells and HT29 cells were then incubated with either truncated murine or truncated human APRIL ligand polypeptides, respectively, produced by the methods disclosed herein (Examples 1-4) at the concentration range from 0 to 50 ng/mL for 10 minutes. Cells were washed in cold PBS and then lysed in lysis buffer (1% Triton X-100, 50 mM Hepes, pH 7.5, 10% glycerol, 100 mM sodium phosphate, 10 mM sodium pyrophosphate, 100 mM sodium fluoride, 3 mM sodium orthovanadate, 50 mM β-glycerol phosphate) containing protease inhibitors (Boehringer Mannheim, Indianapolis, Ind.). Whole cell lysates were placed on ice for 10 minutes and then centrifuged for 15 minutes at 12,000 rpm at 4° C. in an Eppendorf microfuge and supernatants were used for analysis. Equal amounts of proteins were resolved at 125 V on SDS-PAGE gels at a 4-20% gradient and electrophoretically transferred to nitrocellulose membranes (Invitrogen, Carlsbad, Calif.) for 1 h at 100 V. Membranes were blocked for 1 h in PBS-T (phosphate buffered saline-Tween 20) containing 5% nonfat dried milk at room temperature, probed with a primary polyclonal rabbit antibody specific to the phosphorylated IκB (Cell Signaling, Beverly, Mass.) for 1 h at room temperature followed by probing with a horseradish-peroxidase labeled secondary antibody for 1 h at room temperature. The membrane was developed using the Enhanced ChemiLuminescence detection kit (Amersham Pharmacia, Piscataway, N.J.). To confirm equal protein loading, immunoblots were stripped with 62.5 mM Tris-HCl (pH 6.8) and 2% SDS at 50° C. for 30 min and reprobed with polyclonal rabbit actin antibody (Santa Cruz Biotechnology Inc., Santa Cruz, Calif.).

Taken collectively, the truncated human and murine APRIL ligand polypeptides produced by the methods disclosed herein are able to activate the NF-κB signaling pathway in tumor cells, as evidenced by the decrease in IκB protein in the presence of increasing concentrations of truncated APRIL ligand polypeptide. In the murine system, this activity is clearly related to the properties of cell proliferation and survival as seen in the NIH-3T3 proliferation assay (see Example 7). Similar results have been obtained using the truncated human APRIL ligand polypeptide (amino acid residues 115-250 of SEQ ID NO: 6) and HT29 cell proliferation after serum starvation (data not shown). Thus, in both the murine and human systems, the truncated APRIL ligand polypeptides of the invention, encoded by amino acid residues 106-241 of SEQ ID NO: 5 (murine) and amino acid residues 115-250 of SEQ ID NO: 6 (human) are biologically active and are able to enhance cell proliferation and survival via the activation of the NF-κB signaling pathway.

### Example 11

### Treatment of HT29 Colon Tumor Epithelial Cells

The role of APRIL ligand in tumor cell proliferation can be evaluated using cells isolated from colon adenocarcinoma (HT29, ATCC, Rockville, Md.). The cells are passaged in DMEM medium containing 10% FBS until cells reach 70% confluency. The cells are then serum-starved overnight, and stimulated with a fusion protein comprising a truncated human APRIL ligand polypeptide (amino acid residues 115-250 of SEQ ID NO: 6) fused to a toxic fusion partner moiety at a concentration from 100 μg/ml to 10 μg/ml. Cell responsiveness is measured by assays of proliferation, survival, signaling (e.g., IκB degradation), and gene and protein induction.

### Example 12

### Human Peripheral Blood B Cell Proliferation

The potential role that APRIL ligand may play in cell proliferation may also be evaluated in human peripheral blood B cells. B cells are isolated from the blood of human donors or patients and passaged in RPMI medium containing 10% FBS. B cells are stimulated with a primary signal (e.g., anti-IgM or CD40L or LPS) in the presence or absence of purified truncated human APRIL ligand polypeptide (amino acid residues 115-250 of SEQ ID NO: 6) at concentrations from 100 pg/ml to 10 μg/ml. The addition of the APRIL signal to the primary signal induces cell proliferation and/or survival, and induces NF-κB signaling leading to the production of anti-apoptotic proteins (e.g., Bcl-2, Bclx1), and to changes in the expression of cell surface proteins, such as MHCII, FAS, CD21, CD23, and CD16/CD32.

### Example 13

### Treatment of Human Rheumatoid Synovial Cells

The potential role of APRIL ligand in cell proliferation involved in human disease conditions, such as rheumatoid arthritis, may also be evaluated by incubating a fusion protein comprising a truncated human APRIL ligand polypeptide (amino acid residues 115-250 of SEQ ID NO: 6) fused to a toxic fusion partner moiety with cells isolated from the synovial tissue of rheumatoid arthritis patients. The cells are passaged in DMEM medium or RPMI medium containing 10% FBS until cells reach 70% confluency. The cells are then serum-starved overnight, and stimulated with purified truncated human APRIL ligand fusion polypeptide (amino acid residues 115-250 of SEQ ID NO: 6) at concentrations from 100 pg/ml to 10 μg/ml in the presence or absence of additional agent (e.g., IFN-γ, IL-1β, TNF) known to stimulate such cells. Cell responsiveness is measured by assays of proliferation, survival, signaling (e.g., IκB degradation), and gene and protein induction. This stimulation has the properties of being additive or synergistic with other known stimulators of such cells (e.g., IFNγ, IL-1β, TNF).

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the disclosure herein, including the appended claims.

1. A method of producing a biologically active truncated APRIL ligand polypeptide or analog thereof comprising the steps of:
(a) providing a vector comprising a nucleotide sequence encoding a truncated APRIL ligand polypeptide operably linked to an expression control sequence;
(b) introducing the vector into a host cell;
(c) growing the host cell in a culture medium under conditions which allow the truncated APRIL ligand polypeptide or analog thereof to be expressed and secreted into the culture medium;
(d) separating the culture medium from the host cell;
(e) subjecting the separated culture medium to adsorption chromatography; and
(f) recovering truncated APRIL ligand polypeptide or analog thereof fractions.

2. The method according to claim 1, wherein the truncated APRIL ligand polypeptide is human.

3. The method of claim 2, wherein the truncated APRIL ligand polypeptide is selected from the group consisting essentially of amino acid residues ranging from amino acids 115-250 of SEQ ID NO: 6 to amino acids 133-250 of SEQ ID NO: 6.

4. The method of claim 3, wherein the truncated APRIL ligand polypeptide consists essentially of amino acid residues 115-250 of SEQ ID NO: 6.

5. The method according to claim 1, wherein the truncated APRIL ligand polypeptide is murine.

6. The method of claim 5, wherein the truncated APRIL ligand polypeptide consists essentially of amino acid residues 106-241 of SEQ ID NO: 5.

7. The method according to claim 1, wherein the adsorption chromatography is selected from the group consisting of hydroxyapatite chromatography and affinity chromatography.

8. The method according to claim 7, wherein the adsorption chromatography is hydroxyapatite chromatography.

9. The method according to claim 7, wherein the adsorption chromatography is affinity chromatography.

10. The method according to claim 9, wherein the affinity chromatography is carried out using M1 Sepharose.

11. The method according to claim 1, wherein the nucleotide sequence is human.

12. The method according to claim 11, wherein the nucleotide sequence is SEQ ID NO: 7.

13. The method according to claim 1, wherein the nucleotide sequence is murine.

14. The method according to claim 13, wherein the nucleotide sequence is SEQ ID NO: 1.

15. The method according to claim 1, further comprising the step of subjecting the truncated APRIL ligand polypeptide or analog thereof fractions to size exclusion chromatography.

16. The method according to claim 15, wherein the nucleotide sequence is selected from the group consisting of SEQ ID NO: 1 and SEQ ID NO: 7.

17. The method according to claim 15, further comprising the step of subjecting the truncated APRIL ligand polypeptide or analog thereof fractions to ion exchange chromatography.

18. The method according to claim 17, wherein the nucleotide sequence is selected from the group consisting of SEQ ID NO: 1 and SEQ ID NO: 7.

19. The method according to claim 17, wherein the ion exchange chromatography is carried out using SP Sepharose.

20. The method according to claim 15, wherein the size exclusion chromatography uses a size exclusion matrix capable of resolving proteins under 200 kilodaltons.

21. The method according to claim 15, wherein the size exclusion chromatography uses a size exclusion matrix capable of resolving proteins under 100 kilodaltons.

22. The method according to claim 15, wherein the size exclusion chromatography uses a size exclusion matrix capable of resolving proteins under 75 kilodaltons.

23. The method according to claim 15, wherein the size exclusion chromatography is carried out using a size exclusion matrix selected from the group consisting of sephacryl 100, superdex 200 and superdex 75.

24. The method according to claim 1, wherein the vector is derived from the plasmid pIC9.

25. The method according to claim 1, wherein the vector is derived from the plasmid pCR3.

26. The method according to claim 1, wherein said expression control sequence is selected from the group consisting of a viral sequence, a bacterial sequence, a yeast sequence, an insect sequence, and a mammalian sequence.

27. The method according to claim 26, wherein said expression control sequence is a yeast sequence.

28. The method according to claim 27, wherein the expression control sequence is methanol oxidase promoter.

29. The method of claim 1, wherein the host cell is a bacterial, yeast, mammalian, or insect cell.

30. The method according to claim 29, wherein the host cell is a yeast cell.

31. The method according to claim 30, wherein the yeast cell is Pichia pastoris.

32. The method according to claim 29, wherein the host cell is a mammalian cell.

33. The method according to claim 32, wherein said mammalian cell is kidney 293T cells.

34. The method according to claim 1, wherein the truncated APRIL ligand polypeptide or analog thereof is fused to a fusion partner.

35. The method according to claim 34, wherein the fusion partner is a Myc tag.

36. The method according to claim 34, wherein the fusion partner is a FLAG tag.

37. A biologically active truncated APRIL ligand polypeptide or analog thereof produced according to the method of claim 1.

38. A biologically active truncated APRIL ligand polypeptide or analog thereof.

39. The truncated APRIL ligand polypeptide or analog thereof according to claim 37 or 38, wherein the polypeptide is human.

40. The truncated APRIL ligand polypeptide according to claim 39, wherein the polypeptide is selected from the group consisting essentially of:
(a) amino acid residues ranging from amino acids 115-250 of SEQ ID NO: 6 to amino acids 133-250 of SEQ ID NO: 6; and
(b) a polypeptide encoded by a nucleotide sequence of SEQ ID NO: 7.

41. The truncated APRIL ligand polypeptide or analog thereof according to claim 37 or 38, wherein the polypeptide is murine.

42. The truncated APRIL ligand polypeptide according to claim 41, wherein the polypeptide is selected from the group consisting essentially of:
(a) amino acid residues 106-241 of SEQ ID NO: 5; and
(b) a polypeptide encoded by a nucleotide sequence of SEQ ID NO: 1.

43. An isolated nucleic acid molecule comprising a nucleotide sequence encoding a biologically active truncated APRIL ligand polypeptide according to claim 37.

44. A pharmaceutical composition comprising the truncated APRIL ligand polypeptide according to claim 37 or 38 and a therapeutically acceptable carrier, adjuvant or vehicle.

45. The pharmaceutical composition according to claim 44, wherein the composition is formulated for delivery by oral, parenteral, pulmonary, nasal, aural, anal, dermal, ocular, intravenous, intramuscular, intraarterial, intraperitoneal, mucosal, sublingual, subcutaneous, transdermal, topical, sustained release, intracranial, or buccal cavity route.

46. A method for promoting cell proliferation in an immunosuppressed subject comprising the step of administering to the subject a therapeutically effective amount of a pharmaceutical composition according to claim 44.

47. The method according to claim 46, further comprising the step of administering at least one additional agent.

48. The method according to claim 47, wherein the additional agent is selected from the group consisting of IFN-γ, IL-1B and TNF.


SEQUENCE LISTING (11 sequences)

SEQ ID NO: 1 - DNA, 438 bp, Mus sp.
cactcagtcctgcatcttgttccagttaacattacctccaaggatcttgttccagttaacattacctccaaggactctgacgtgacagaggtgatgtggcaaccagtacttaggcgtgggagaggcctggaggcccagggagacattgtacgagtctgggacactggaatttatctgctctatagtcaggtcctgtttcatgatgtgactttcacaatgggtcaggtggtatctcgggaaggacaagggagaagagaaactctattccgatgtatcagaagtatgccttctgatcctgaccgtgcctacaatagctgctacagtgcaggtgtctttcatttacatcaaggggatattatcactgtcaaaattccacgggcaaacgcaaaacttagcctttctccgcatggaacattcctggggtttgtgaaactatga

SEQ ID NO: 2 - DNA, 33 bp, Mus sp.
tgtggagctccactcagtcctgcatcttgttcc

SEQ ID NO: 3 - DNA, 33 bp, Mus sp.
tgtgcggccgctcatagtttcacaaaccccagg

SEQ ID NO: 4 - protein, 135 aa, Mus sp.
HSVLHLVPVNITSKDSDVTEVMWQPVLRRGRGLEAQGDIVRVWDTGIYLLYSQVLFHDVTFTMGQVVSREGQGRRETLFRCIRSMPSDPDRAYNSCYSAGVFHLHQGDIITVKIPRANAKISLSPHGTFLGFVKL

SEQ ID NO: 5 - protein, 241 aa, Mus sp.
MPASSPGHMGGSVREPALSVALWLSWGAVLGAVTCAVALLIQQTELQSLRREVSRLQRSGGPSQKQGERPWQSLWEQSPDVLEAWKDGAKSRRRRAVLTQKHKKKHSVLHLVPVNITSKADSDVTEVMWQPVLRRGRGLEAQGDIVRVWDTGIYLLYSQVLFHDVTFTMGQVVSREGQGRRETLFRCIRSMPSDPDRAYNSCYSAGVFHLHQGDIITVKIPRANAKLSLSPHGTFLGFVKL

SEQ ID NO: 6 - protein, 250 aa, Homo sapiens [sequence omitted]

SEQ ID NO: 7 - DNA, 408 bp, Homo sapiens
cactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 8 - DNA, 1348 bp, Homo sapiens
ggtacgaggcttcctagagggactggaacctaattctcctgaggctgagggagggtggagggtctcaaggcaacgctggccccacgacggagtgccaggagcactaacagtacccttagcttgctttcctcctccctcctttttattttcaagttcctttttatttctccttgcgtaacaaccttcttcccttctgcaccactgcccgtacccttacccgccccgccacctccttgctaccccactcttgaaaccacagctgttggcagggtccccagctcatgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtgattgtgttataaaaagtggctcccagcttggaagaccagggtgggtacatactggagacagccaagagctgagtatataaaggagagggaatgtgcaggaacagaggcatcttcctgggtttggctccccgttcctcacttttcccttttcattcccaccccctagactttgattttacggatatcttgcttctgttccccatggagctccgaattcttgcgtgtgtgtagatgaggggcgggggacgggcgccaggcattgttcagacctggtcggggcccactggaagcatccagaacagcaccaccatctta

SEQ ID NO: 9 - DNA, 34 bp, Homo sapiens
tgtgtctgcagcactctgtcctgcacctggttcc

SEQ ID NO: 10 - DNA, 33 bp, Homo sapiens
tgtgtgggccctcacagtttcacaaaccccagg

SEQ ID NO: 11 - protein, 136 aa, Homo sapiens
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL